(19) United States
(12) Patent Application Publication
(10) Pub. No.: US 2008/0311061 A1
(43) Pub. Date: Dec. 18, 2008

(54) Cosmetic Skin Color Determination and Color Mixing Apparatus

(76) Inventor: Sigrid Heuer, Frankfurt (DE)

Correspondence Address: DARBY & DARBY P.C., P.O. BOX 770, Church Street Station, New York, NY, 10008-0770, US

(21) Appl. No.: 11/574,443

(22) PCT Filed: Aug. 29, 2005
(86) PCT No.: PCT/IB2005/003569

§ 371 Date: Feb. 28, 2007

(30) Foreign Application Priority Data
Aug. 30, 2004 (DE) 10 2004 041 814.4

(51) Int. Cl. A61K 8/00; B01F 13/10

(52) U.S. Cl. 424/63; 366/142

(57) ABSTRACT

A cosmetic color apparatus includes a color measurement device for measuring a skin color of an individual. A computer processing device is provided in communication the color measurement device for performing a comparison of the measured skin color with a desired color so as to provide color information. A color mixing device is provided in communication with the computer processing device for mixing a plurality of pigments based on the color information so as to provide a cosmetic color mixture.

The present invention relates to cosmetic color determination and preparations.

### BACKGROUND

In cosmetic applications, customers choose from a variety of fixed colors to be put on the skin (decorative cosmetics and make-up) or under the skin (permanent make-up and tattoos). The process of simply choosing a color for permanent make-up/tattoos or decorative cosmetics/make-up purposes does not take into account the individual skin pigmentation, or skin color, of the customer.

This practice of not taking into account the customer's skin pigmentation has a negative effect on the final color outcome. Ignoring the natural skin pigmentation, whose individual pigments may not be easily visible to the human eye, and therefore, not mixing pigments that will accommodate the pigments of the skin, can lead to undesirable and uncorrectable color results especially in the permanent make-up segment.

For example, a blue color applied to the skin may appear greenish, depending upon the amount of yellow pigment in the skin. Red, depending on the yellow pigment in the skin, may appear orange. By not taking the basic skin pigmentation of the person into account, a blue eyeliner applied under skin having yellow skin pigmentation will appear green sooner or later, or may look greenish from the outset. Incorporating the given skin pigmentation in the determination/mixing of the desired color is therefore advantageous.

Moreover, color changes can occur in skin where permanent make-up/tattoos have been applied, potentially resulting in undesirable apparent color changes.

Questionable “color stabilization chemicals” in the colors sold to date, among other things, are applied to work around such influencing factors as the natural skin pigmentation, as well as sun and enzyme reactions. The health-related side effects of permanent make-up/tattoos have been the topic of public discussions in Europe for many years.

Colors can be numerically presented in different calorimetric systems, or standard color spaces, for example, CIEXYZ or CIELAB, as described in CIE (Commission Internationale de L'Éclairage) Publication 15.3, which is hereby incorporated by reference herein.

Software programs, such as “Color Master Software” from the X-Rite corporation, which software program is hereby incorporated by reference herein, are available for performing color formulation and color difference/quality assurance tasks.

### SUMMARY OF THE INVENTION

The present invention provides a cosmetic color apparatus including a color measurement device configured to measure a skin color of an individual. A computer processing device is provided in communication the color measurement device and configured to perform a comparison of the measured skin color with a desired color so as to provide color information. A color mixing device is provided in communication with the computer processing device and configured to mix a plurality of pigments based on the color information so as to provide a cosmetic color mixture.

The color measurement device may be a spectrophotometer. The color information may include a recipe of respective amounts of a plurality of pigments based on a difference between the measured skin color and the desired color.

The present invention also provides a method for providing a cosmetic color mixture. The method includes:

measuring a skin color of an individual;

comparing the measured skin color with a desired color so as to provide color information; and

mixing a plurality of pigments based on the color information so as to provide a cosmetic color mixture.

Also provided by the present invention is a computer readable medium having stored thereon computer executable process steps operative to perform a method for providing a cosmetic color mixture. The method includes:

measuring a skin color of an individual;

comparing the measured skin color with a desired color so as to provide color information; and

mixing a plurality of pigments based on the color information so as to provide a cosmetic color mixture.

The present invention makes an accurate color determination possible by measuring the natural or pre-existing customer pigmentation and taking this into account for mixing a cosmetic mixture needed to reach a final desired color when applied.

Existing discolorations in skin to which color has already been applied can, using the present invention, be treated with an exactly measured correction color. The cosmetician is thus able to professionally re-pigment colors which have faded over time for whatever reason. Faces that show color fluctuations (red eyebrows) resulting from, for example, sunshine and/or enzyme reactions, can be successfully corrected using the present invention.

### BRIEF DESCRIPTION OF THE DRAWINGS

FIG. 1 shows a schematic block diagram of a cosmetic color apparatus.

FIG. 2 shows use of a spectrophotometer for measuring the skin color of an individual.

FIG. 3 shows sample chart of desired colors.

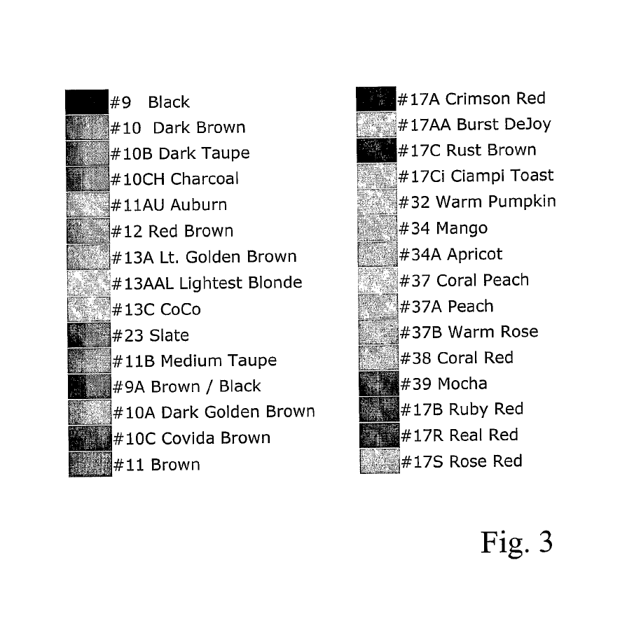

FIG. 4 shows a schematic diagram of a color mixing device.

FIG. 5 shows a portion of a display like that generated by a graphical user interface provided by the “Color Master Software” of the X-Rite corporation, showing an output of a skin color measurement (curve A) and a selected desired color (curve B) graphically as reflectance (in percent) versus wavelength (in nanometers).

### DETAILED DESCRIPTION

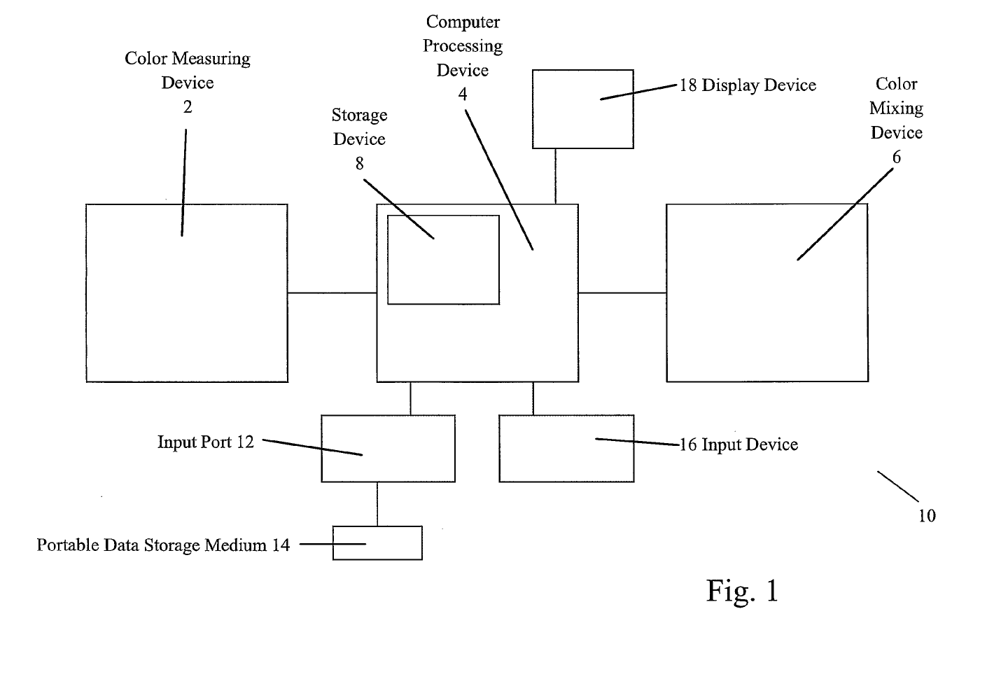

FIG. 1 shows a schematic block diagram of a cosmetic color apparatus 10 according to the present invention. Color measuring device 2 is connected to computer processing device 4, which is in turn connected to color mixing device 6. In an embodiment, computer processing unit 4 is co-located with color measuring device 2 and color mixing device 6, the three devices being connected by appropriate interfaces. In an embodiment, color measuring device 2, computer processing device 4 and color mixing device 6 are interconnected wirelessly.

Color measuring device 2 is used to measure the skin color, or pigmentation, of a person's skin. In an embodiment, color measuring device 2 is a spectrophotometer, such as tethered spectrophotometer model DTP22, from X-Rite corporation. In other embodiments, color measuring device 2 may be a battery-operated unit in wireless communication, using a Bluetooth standard, for example, with computer processing device 4.

In order to measure a color, color measuring device 2 is initially calibrated to standard white. In other embodiments another color may be used for calibration. Then color measurements can begin. Color measuring device 2 is placed on the skin area to be measured and, when activated, emits light. The light reflected from the skin passes through a lens of the measuring device onto a spectral sensor. The spectral sensor measures the intensity of the reflected light at certain wavelengths. The spectral data, or reflectance data, output by the sensor are transmitted to computer processing device 4. The spectral data can be numerically presented in different colorimetric systems, or standard color spaces, for example, CIEXYZ or CIELAB, as described in CIE (Commission Internationale de L'Éclairage) Publication 15.3. For example, the spectral data may be represented as a point defined by a set of coordinates in the color space used.

Computer processing device 4 accepts measured skin color data from color measuring device 2, and determines color information, such as a spectral curve and/or a numerical color value in a predetermined color system, for use by color mixing device 6. In an embodiment, computer processing device 4 is an electronic computing device, such as a personal computer, capable of carrying out programmed instructions using input from color measuring device 2 and desired colors stored as mathematical representations on device 4. The programmed instructions and desired color representations are stored in storage device 8 of computer processing device 4. The programmed instructions are designed to use the input and the mathematically programmed desired colors to determine a difference between the measured skin color and a desired color. Thus, the programmed instructions output color information in the form of a “color delta” that is needed to reach the desired color. The color delta can be represented by, for example, the difference between two points, or coordinate sets, in a color space, or calorimetric system. The programmed instructions compute a “recipe” of respective amounts (for example in grams, millilitres, etc.) of color pigments to be mixed together to produce the color delta.

In an embodiment, computer processing device 4 is a Windows 2000 or XP capable personal computer with 5 MB RAM, and a central processing unit capable of 1 GHz. Storage device 8 may be, for example, a 10 gigabyte hard drive.

The programmed instructions executed by computer processing device 4 consist in an embodiment of a software application interface, for example Win32-API, written in an object oriented language, such as Visual C++ using Microsoft Foundation Classes “MFC” as well as Assembler. The programmed instructions include instructions for processing data from color measuring device 2. In an embodiment, the programmed instructions include the “Color Master Software” from the X-Rite corporation. In other embodiments, other types of programming languages and/or software programs may be employed, for processing data from color measuring device 2, for determining the difference (“color delta”) between the measured skin color and a desired color, and for providing a color recipe to and/or controlling color mixture device 6. In an embodiment, storage device 8 holds color formulas for calculating the color delta. In an embodiment, the calculated color delta may be modified based on the general skin tone or racial type of the person whose skin was measured.

A color, which has been chosen by the customer from a color palette, may be represented in the programmed instructions mathematically as reference values with optical data from which recipes can be computed. Stored mathematical color formulas or coordinates of the calorimetric system represent a desired color. The programmed instructions accept the selected color in the form of, for example, coordinates in a color space, and calculate the color delta to be mixed, i.e., the color needed with reference to the pigments in the skin to reach the desired color. Appropriate mix instructions are accordingly sent to color mixing device 6. The mix instructions include a recipe of respective amounts of color pigments to be mixed together to produce the color delta. In other embodiments of the present invention, a color delta is sent to the mixing device, which itself determines the recipe needed to achieve the color delta.

Input port 12 connected to computer processing device 4 is provided for receiving data input via portable data storage medium 14. Input port 12 may, for example, be a memory disk slot, a USB port, or a CD/DVD/RW unit. Input device 16 connected to computer processing device 4 is provided to enable manual inputting of information into the processing device. Input device 16 may include a keyboard and/or a mouse. Display device 18 connected to computer processing device 4 is provided to output information for viewing by users, such as cosmeticians and cosmetics customers. In an embodiment, display device 18 displays a graphical user interface, and may offer touch-screen or oral input capability.

Color mixing device 6 receives color information, such as a recipe of respective amounts of color pigments to be mixed, from computer processing device 4 and provides a cosmetic color mixture to be applied to an individual's skin to achieve a desired color effect on or in the skin. Color mixing device 6 mixes appropriate amounts, according to the recipe, of pigments, or colors, to provide the intended cosmetic color mixture, or “color delta.”

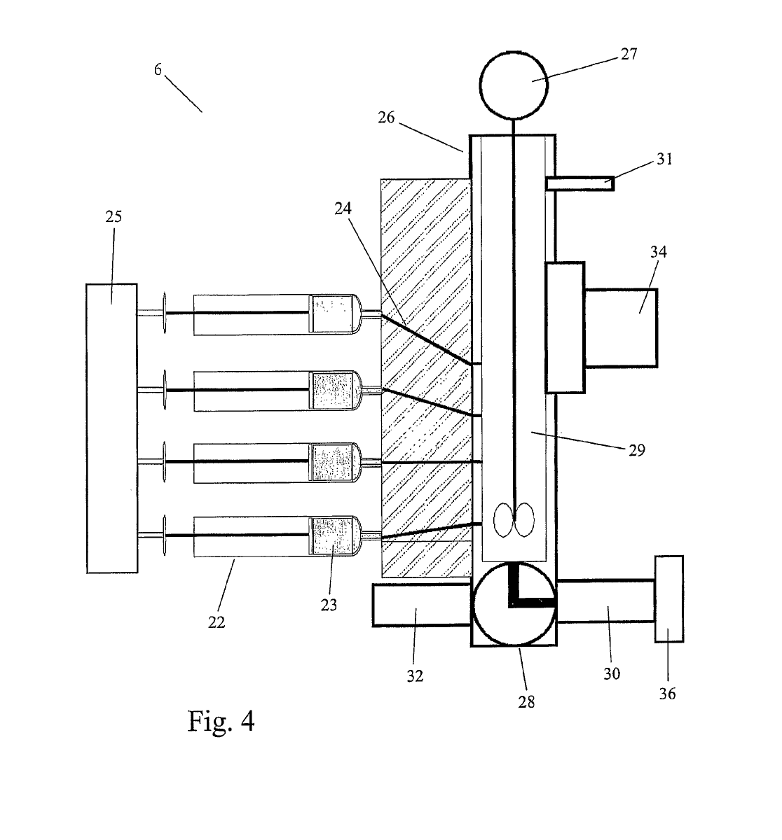

Referring to FIG. 4, in an embodiment color mixing device 6 includes pigment syringes 22, which deliver pigments to mixing vessel 26 via pigment delivery tubes 24. Screw jack motor 25 or other linear actuator device may be used to actuate pigment syringes 22, either individually or in sets. In an embodiment, liquid pigment is delivered with 0.01 mm accuracy. Each pigment syringe 22 contains a different pigment 23 that can flow through a respective pigment delivery tube 24. In an embodiment, pigments 23 are liquid pigments. In other embodiments, pigments 23 are powdered pigments. Four pigment syringes 22 are shown in FIG. 4, though more or fewer syringes may be used depending on the cosmetic/make-up application and other factors.

In an embodiment a minimum of seven pigment syringes 22 are employed in the mixing of the cosmetic color mixture for permanent make-up and decorative cosmetic procedures. For the production of make-up, in an embodiment additional syringes with different color pigments are used. Pigment syringes 22 can hold different concentrations of pigments depending on requirements and fashion. Pigment syringes 22 are in an embodiment used until empty, or until a desired fullness level is reached, and then replaced. In other embodiments, pigment syringes 22 are exchanged after each mixing procedure, depending on which type of screw jack motor or other device is used to actuate the syringes for injecting the pigments into the mixer. Pigment syringes 22 can be located next to each other in a row, or in another embodiment held in a rotating carousel. Pigments 23 can be injected in series using a single screw jack motor, or in other embodiments each pigment syringe can be actuated by its own screw jack motor 25.

Mixing vessel 26 includes mixing unit 27, which may include one or more screws or paddles for pigment mixing, and includes overflow and air vent 31 and three-way valve 28. Three-way rotary valve 28 is positionable to operatively connect mixing vessel 26 to output vent 30 for delivering cosmetic color mixture 29 through the output vent, or to regress/cleaning vent 32 for cleaning of the mixing vessel. Color measurement cell 34 is provided for measuring the color of the color mixture 29 in mixing vessel 26. In an embodiment, color measuring cell 34 is a spectrophotometer supported by a cuvette. In an embodiment, color measurement cell 2 may be used to confirm or control color mixture 29.

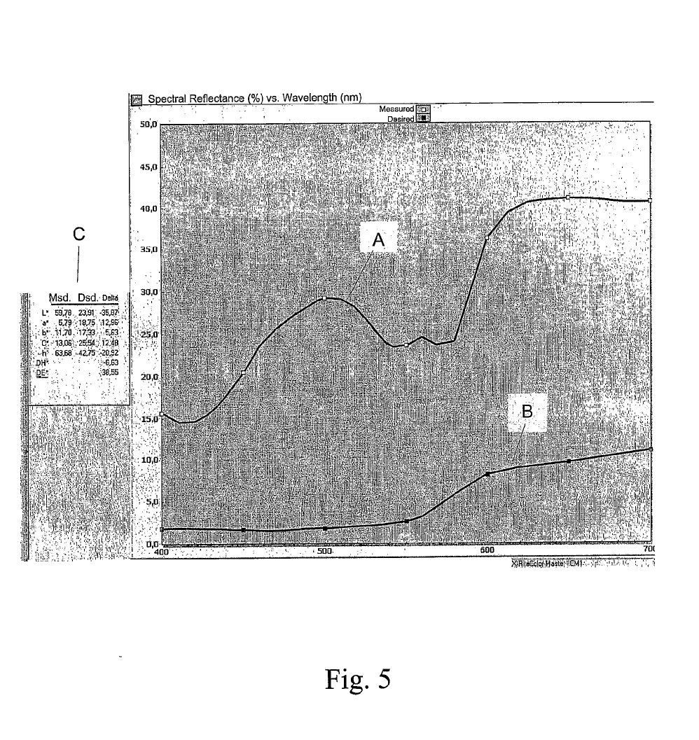

As noted above, in an embodiment of the present invention, the programmed instructions executed by computer processing device 4 include the “Color Master Software” from the X-Rite corporation. FIG. 5 shows a portion of a display like that generated by a graphical user interface provided by the “Color Master Software.” The display shows a graph of reflectance (in percent) versus wavelength (in nanometers). Curve A represents an output of a skin color measurement. Curve B represents the desired color selected by the customer. The “Color Master Software” calculates the difference between the measured and selected colors, producing a “color delta” to be mixed by color mixing device 6. Column C in FIG. 5 shows the color space coordinates of the measured skin color (“Msd.”), the desired color (“Dsd.”), and the color delta (“Delta”).

### Example Permanent Cosmetic or Make-Up Process

A process according to the present invention for providing a cosmetic color mixture for a permanent cosmetic or make-up will now be described:

The user, for example a cosmetician, begins the session by turning on computer processing device 4, in this case a personal computer. A first window appearing on display device 18 gives the user the choice between permanent cosmetic, decorative cosmetic, and make-up. Clicking the permanent cosmetic brings the user to a window with the following options: “New Customer” and “Existing Customer.”

“New Customer”—The user is prompted to input, into input port 12, a portable data storage medium 14, such as a customer card, mini CD, memory stick, etc. The portable storage medium is used to save the customer's personal data as well as the data with reference to the visit. The user inputs, name, address, and treatment, including color pigment data. Portable storage medium 14 is left in the input port 12 until the session has ended. Data is saved on the portable storage medium, which is given to the customer after the session.

“Existing Customer”—This option loads the data from the customer's last visit and makes this available to the user. The user is prompted to input portable data storage medium 14, which contains the pre-existing data. At this point the user can update any changes. In any case, the system is now available for use and the portable storage medium is updated with the steps taken, saved, copied and given to the customer.

The user can choose between the options: Eyebrow, Lips, Eye Liner, Make-up, etc.

The procedure is as follows:

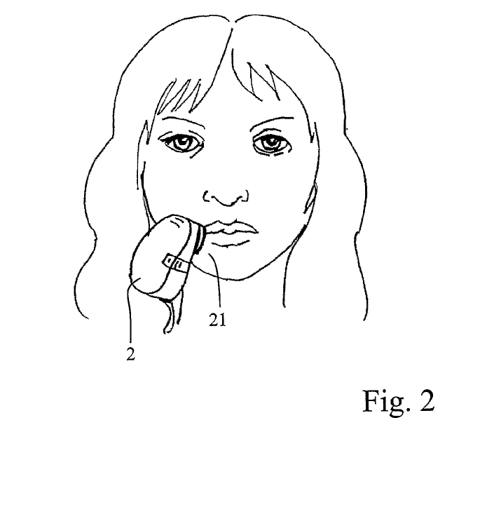

The user clicks on the icon “Measure” shown on display device 18. Clicking on “Measure” activates the pigment measuring function. The user places color measuring device 2, for example a handheld spectrophotometer, on area 21 that is to be treated, as shown in FIG. 2. On the handheld unit is a button or another mechanism that when pressed or otherwise activated, measures the color, or pigmentation, of the skin of the area to be treated. The color, indicated as one or more numbers, a line, a graph or otherwise, is then presented on the display device. In an embodiment the color is represented by one or more wavelength values, or another number or numbers.

It should be noted that prior to the measurement procedure, depending on the customer's skin structure and in order to achieve an exact measurement, in an embodiment the customer's skin is prepared using a vacuum attachment, by injection, etc., in order to smooth or flatten the skin area to be measured.

The user can repeat this measurement procedure at different points on the area to be treated. The user can, depending on the software used, click on an “Average” icon and an average color value of the measured color values is calculated.

The customer then chooses a desired color from a displayed chart or palette of available colors. FIG. 2 shows a sample chart of available colors, each color being identified by a respective reference number. The user inputs the relevant number into computer processing device 4 via input device 16, by typing or clicking the color on display device 18.

The user then clicks on an icon “Calculate” shown on display device 18. This prompts computer processing device 4 to calculate the “color delta,” i.e., the difference between the measured skin color and the selected desired color. The color delta represents the color that will be pigmented in the skin of the customer (or used to make decorative make-up). Computer processing device 4 also determines a recipe of respective amounts of pigments to achieve the color delta, and sends the recipe to color mixing device 6 via an appropriate interface.

In an embodiment, storage device 8 holds a databank of 10 or more variations or shades of various colors representing predetermined color differences. When the color delta has been determined, computer processing device 4 searches the databank and identifies the saved color shade (predetermined color difference) that is the closest to the determined color delta. Computer processing device 4 sends a recipe for the identified closest color shade to color mixing device 6 via an appropriate interface.

When display device 18 indicates that color mixing device 6 is “Ready,” displaying a corresponding icon, the user then clicks on the icon “Mix” shown on the display device, thereby activating screw jack motor 25 of mixing unit 27 of the mixing device. The required pigments are injected via pigment delivery tubes 24 into mixing vessel 26 to reach the color delta. Color measurement cell 34 is used by computer processing device 4 to verify that the color delta has been reached, and if the color delta is not reached a control activates a metered addition of one or more needed pigments 23 until the color delta is reached. Three-way rotary valve 28 is positioned to provide cosmetic mixture 29 to output vent 30 for delivery to container 36.

In an embodiment, as a visual check, the desired color is shown on display device 18 and labelled as “desired color.” Once the color delta is calculated the color delta and the captured skin color are electronically mixed and shown next to the “desired color” and labelled as “suggested mix.” If the “suggested mix” is acceptable, the “mix” icon is clicked, computer processing device 4 produces a recipe for the color delta and sends the recipe to color mixing device 6. If the suggested mix is not correct, the color delta can be either recalculated or adjusted to achieve a new suggested mix. Clicking the “mix” icon sends an appropriate recipe to the color mixing device 6.

If mixing device 6 has not been used within the past day, computer processing device 4 sends a “Clean” signal to the mixing device, and before the color delta is mixed a cleaning process is run. For the cleaning process, three-way rotary valve 28 is positioned to connect mixing vessel 26 to regress/cleaning vent 32. A cleaning solution (in an embodiment, 70% isopropyl alcohol and 30% water) is released into mixing vessel 26, and mixing unit 27 is activated. Computer processing device 4 uses color measurement cell 34 to verify that mixing vessel 26 is clean. The cleaning process is repeated until the mixing vessel is clean. Then the cleaning solution is discharged via regress/cleaning vent 32, possibly with the aid of compressed air applied via overflow and air vent 31. Three-way rotary valve 28 then assumes a neutral position. When the cleaning process is complete the “Ready” icon is activated.

The user takes container 36 from mixing device 6 and performs the skin pigmentation procedure. In an embodiment, the skin pigmentation procedure is performed using a permanent make-up machine to pigment the color delta under the customer's skin. The permanent make-up machine may be a battery driven or plug-in handheld device usable with different needles.

After the skin pigmentation procedure is completed, including at a later date, color measurement device 2 may be used together with computer processing device 4 to verify that the applied color is as desired.

If the color measured on the skin matches the desired color, then the user clicks a “Save and Exit” icon presented on display device 18. Computer processing device 4 saves in storage device 8 under the customer's name the information generated for this customer during the session. Computer processing device 4 also saves, on portable data storage medium 14 of the customer, the color delta mixture calculation for the skin area measured or treated during the session.

The process for decorative cosmetics is similar to that for permanent make-up. A difference lies in the fact that the cosmetic color mixture (color delta) produced is mixed into base products for liquid eyeliner, liquid eyebrow liner, liquid lipstick, etc.

The process for make-up is also similar to that for permanent make-up, although in some embodiments additional syringes with different pigments are needed. The cosmetic color mixture produced is mixed into base products for make-ups.

While the invention has been described in connection with individual embodiments, it is not intended to limit the scope of the invention to the particular form set forth, but, on the contrary, it is intended to cover such alternatives, modifications and equivalents as may be included within the spirit and scope of the invention as defined by the appended claims. For example, other types of color mixing devices may be employed within the scope of the invention. In an embodiment, a membrane pump is used to pump a mixture of appropriate pigments through a static potential energy mixer until a color measuring cell determined that a desired color mixture has been achieved. In another embodiment, an electromagnetic power compression measuring scale is used to measure specific amounts of pigments delivered in measured volumes by respective pumps activated in sequence.

1: A cosmetic color apparatus comprising;
a color measurement device configured to measure a skin color of an individual;
a computer processing device in communication with the color measurement device and configured to perform a comparison of the measured skin color with a desired color so as to provide color information; and
a color mixing device in communication with the computer processing device and configured to mix a plurality of pigments based on the color information so as to provide a cosmetic color mixture.

2: The apparatus as recited in claim 1 wherein the color measurement device includes a spectrophotometer.

3: The apparatus as recited in claim 1 wherein the color information includes a recipe of respective amounts of a plurality of pigments, the recipe being based on a difference between the measured skin color and the desired color.

4. The apparatus as recited in claim 1 wherein the comparison includes:
determining a difference between the measured skin color and the desired color based on the comparison; and
selecting, from a plurality of stored desired color difference values, a desired color difference that is closed to the determined difference.

5: The apparatus as recited in claim 1 wherein the computer processing device is configured to provide the color information based on a skin tone or racial type of the individual.

6. The apparatus as recited in claim 1 wherein the computer processing device is configured to store at least one of a plurality of predetermined desired color values and a plurality of predetermined color difference values.

7: The apparatus as recited in claim 1 wherein the computer processing device is configured to at least one of read and store information of the individual on a portable computer readable storage medium.

8. The apparatus as recited in claim 1 wherein the computer processing device is configured to select the desired color.

9. The apparatus as recited in claim 1 wherein the color mixing device includes a screw jack motor.

10: The apparatus as recited in claim 1 wherein the cosmetic color mixture is at least one of a decorative cosmetic mixture, a make-up mixture, a permanent cosmetic mixture and a tattoo mixture.

11. A method for providing a cosmetic color mixture, the method comprising:
measuring a skin color of an individual;
comparing the measured skin color with a desired color so as to provide color information; and
mixing a plurality of pigments based on the color information so as to provide a cosmetic color mixture.

12: The method as recited in claim 11 wherein the measuring is performed using a spectrophotometer.

13: The method as recited in claim 11 wherein the color information includes a recipe of respective amounts of a plurality of pigments, the recipe being based on a difference between the measured skin color and the desired color.

14: The method as recited in claim 1 wherein the comparing includes:
determining a difference between the measured skin color and the desired color based on the comparison; and
selecting, from a plurality of stored desired color difference values, a desired color difference that is closed to the determined difference.

15: The method as recited in claim 11 wherein the comparing is performed using a computer processing device.

16: The method as recited in claim 15 further comprising storing at least one of a plurality of predetermined desired color values and a plurality of predetermined color difference values using the computer processing device.

17. The method as recited in claim 15 further comprising at least one of reading and storing information of the individual on a portable computer readable storage medium using the computer processing device.

18. The method as recited in claim 15 further comprising selecting the desired color using the computer processing device.

19. The method as recited in claim 11 wherein the cosmetic color mixture is at least one of a decorative cosmetic mixture, a make-up mixture, a permanent cosmetic mixture and a tattoo mixture.

20: A computer readable medium having stored thereon computer executable process steps operative to perform the method for providing a cosmetic color mixture as recited in claim 11.

21. A cosmetic color apparatus comprising;
a computer processing device configured to provide color information; and
a color mixing device in communication with the computer processing device and configured to mix a plurality of pigments based on the color information so as to provide a cosmetic color mixture.

22. The apparatus as recited in claim 21 wherein the computing device is configured to provide the color information based on a skin tone or racial type.
